(19) United States
(12) Patent Application Publication
(10) Pub. No.: US 2005/0209669 A1
(43) Pub. Date: Sep. 22, 2005

(54) System for stent placement in a vasculature bifurcation

(76) Inventor: John A. Kao, La Jolla, CA (US)

Correspondence Address: Attn: NEIL K. NYDEGGER;NYDEGGER & ASSOCIATES, 348 Olive Street, San Diego, CA, 92103, US

(21) Appl. No.: 10/791,975

(22) Filed: Mar. 3, 2004

(51) Int. Cl. A61F 2/06

(52) U.S. Cl. 623/1.11

(57) ABSTRACT

A positioning system for use in placing a stent at a vascular bifurcation includes an elongated catheter having an inflatable balloon mounted at its distal end. With the balloon deflated, the catheter/balloon assembly is engageable with the stent allowing the stent to be advanced through the vasculature to the bifurcation. At the bifurcation, the balloon can be selectively inflated to release the stent. A plurality of individually activatable, acoustic transducer crystals is mounted on the catheter at the proximal end of the stent. Each crystal can be selectively activated from an extracorporeal location to radiate an acoustic signal that is directed toward a vessel wall where it is reflected, creating an acoustic return signal. The return signal is then received by the respective transmitting crystal and converted to an electrical signal. The electrical signals are then used to determine a spatial relationship between the stent and the bifurcation.

### FIELD OF THE INVENTION

The present invention pertains generally to medical catheters. More particularly, the present invention pertains to medical catheters for stenting a collapsed or stenosed vessel. The present invention is particularly, but not exclusively, useful as a catheter for positioning and implanting a stent at a bifurcation in the vasculature of a patient.

### BACKGROUND OF THE INVENTION

The percutaneous treatment of coronary bifurcation lesions continues to pose a number of technical challenges to practicing cardiologists in spite of recent advances in the treatment of ordinary vascular lesions. A coronary bifurcation, which is typically characterized as having a main branch and a side branch, is inherently more complicated to treat than an ordinary vascular lesion, in part because treatments conducted on the main branch can have adverse consequences on the side branch, and vice versa. For example, a common approach to treating bifurcation lesions is to implant a tubular metallic prosthesis (i.e. stent) in the main branch of the bifurcation across the ostium of the side branch. However, unlike the stenting of an ordinary vessel, the implantation of a stent within the main branch of a bifurcation must be accomplished while maintaining blood flow through the side branch and ensuring that the side branch is accessible to facilitate subsequent percutaneous intervention in the side branch.

Various stenting schemes have been developed to revascularize collapsed or dangerously stenosed coronary bifurcations. These various schemes involve the implantation of one or more stents in the main branch, side branch, and in a majority of cases, stents are implanted in both branches. As indicated above, stents are sometimes implanted in the main branch across the ostium of the side branch. In these cases, specialized stents are sometimes used having a lateral opening midway between the stent ends. During implantation, it is critical that this lateral opening be positioned at and oriented toward the ostium of the side branch. On the other hand, during the implantation of a side branch stent, it is important to position the proximal end of the stent in the side branch, and typically, in the vicinity of the ostium. Portions of the side branch stent which project into the main branch can interrupt main branch flow and prevent a subsequent main branch intervention.

For all the cases discussed above, it is crucial that the stent be positioned accurately prior to implantation. Typically, stents are delivered to the vascular bifurcation and implanted using an inflatable balloon. Once the stent has been implanted, it is extremely challenging to dislodge and remove the stent. Even in cases where the improperly positioned stent is successfully removed, the removal procedure often results in tissue scarring and restenosis, aggravating the initial condition that precipitated the intervention. In addition, the more recent use of drug-eluting stents underscores the notion that accurate stent placement is important. Specifically, it is desirable that these drug-eluting stents be accurately located relative to target tissue to ensure that medicament released from the stent reaches the targeted tissue.

For all vascular bifurcation stenting procedures, the prescribed stent location is often dependent on the bifurcation geometry and the size and location of the lesion(s). Anatomically, bifurcation geometry can vary over a relatively large range with some side branches projecting from their respective main branch at relatively small angles, while other bifurcations have side branches that offshoot from the main branch at angles that are close to ninety degrees. In addition, the three dimensional nature of some bifurcations, wherein the main branch and side branch are not collocated in the same plane, can also make stent positioning a challenge for the practicing cardiologist. The use of external radiography, which typically presents the bifurcation as a two dimensional image, is often suboptimal for use in deciphering the three dimensional nature of the vascular bifurcation. Further compounding these positioning challenges is the fact that the vessels of the bifurcation reside in a “living body environment,” and, as a consequence, are constantly moving as the patient breathes and the patient's heart beats.

The present invention recognizes that useful information regarding the anatomy of a bifurcation including the presence and locations of any lesions can be obtained using acoustic energy that is generated in the vasculature near the bifurcation. In particular, diagnostic ultrasound techniques can give information about tissue condition by differentiating between tissues at their anatomic boundaries inside the patient. Specifically, this happens as transmitted ultrasound waves reflect back to the transducer from these boundaries. The amplitude of the reflected ultrasound waves is then displayed as different shades of gray. Thus, anatomic structures having different acoustic densities will be portrayed with different levels of brightness. Moreover, this technology can be used to determine the bifurcation anatomy relative to the stent to allow for the proper positioning of the stent.

In light of the above, it is an object of the present invention to provide systems and methods suitable for the purposes of accurately positioning and implanting a stent at a vascular bifurcation. It is another object of the present invention to provide systems and methods for mapping the anatomical geometry of a bifurcation and the size/location of any lesions, relative to a stent that has been positioned in the patient's vasculature, to assist the cardiologist in the proper placement of the stent. Yet another object of the present invention is to provide systems and methods for positioning a stent at a bifurcation which are easy to use, relatively simple to implement, and comparatively cost effective.

### SUMMARY OF THE INVENTION

The present invention is directed to a positioning system for use in placing a stent at a bifurcation in the vasculature of a patient. The positioning system includes an elongated catheter that defines a longitudinal axis and has an inflatable balloon mounted at its distal end. The balloon is reconfigurable on the catheter between a first, deflated configuration and a second, radially expanded (i.e. inflated) configuration. Functionally, the catheter/balloon assembly is engageable with the stent when the balloon is in the first configuration, allowing the balloon and stent to be advanced through the vasculature to the site of the bifurcation. At the vascular bifurcation, the balloon can be selectively inflated to release the stent from the balloon.

Prior to a balloon inflation, the system allows the stent to be precisely positioned at the correct location within the bifurcation. To this end, the positioning system includes a plurality of individually activatable, acoustic transducer crystals. Typically, each crystal is mounted on the catheter at the proximal end of the stent. In a particular embodiment, the plurality of crystals are arranged as an annulus (or partial annulus) that is aligned with a plane that is substantially perpendicular to the catheter axis. For the positioning system, each crystal can be selectively activated from an extracorporeal location to radiate an acoustic signal, such as an ultrasonic signal, that is directed substantially radially from the catheter axis.

With the above-described cooperation of structure, the acoustic signal from each transducer crystal is transmitted toward a vessel wall where it is reflected (as an acoustic return signal). The acoustic return signal is then received by the respective transmitting transducer crystal and converted to an electrical return signal. The electrical return signal from each transducer crystal is sent over a wire to an extracorporeally located display where the electrical return signals are used to determine a spatial relationship between the stent and the bifurcation. In some implementations, the electrical return signals can be used to evaluate the nature of the vessel wall, including the presence/absence of a lesion.

In a typical procedure, the stent is engaged with the balloon and advanced through the vasculature to a location near the target bifurcation. In most cases, a guidewire is used for this purpose. Once the stent is near the target bifurcation, the transducer crystals are activated resulting in the generation of electrical return signals, as described above. Next, the catheter is advanced through a small axial distance, and again, the transducer crystals are activated and the resultant return signals analyzed. This procedure is then repeated until the operator is satisfied that the stent is located at the correct position. With the stent correctly positioned, the balloon is inflated to implant and release the stent. The balloon is subsequently deflated and removed from the patient to complete the procedure.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a simplified, perspective view of a positioning system for use in placing a stent at a bifurcation in the vasculature of a patient, shown with a distal portion of the system operationally positioned in the upper body of a patient;

FIG. 2 is an enlarged side view of the distal portion of the system shown in FIG. 1 positioned proximal to a vascular bifurcation, with the bifurcation branches shown in cross-section as seen along line 2-2 in FIG. 1, for clarity;

FIG. 3 is a cross-sectional view of a catheter tube as would be seen along line 3-3 in FIG. 2, showing the guidewire and inflation lumens;

FIG. 4 is a cross-sectional view of a transceiver unit as would be seen along line 3-3 in FIG. 2, showing the individual acoustic transducers;

FIG. 5 is an enlarged side view as in FIG. 2, shown after a distal movement of the balloon and stent;

FIG. 6 is an enlarged side view as in FIG. 2, shown with the balloon in an inflated configuration to release and implant the stent; and

FIG. 7 is an enlarged side view as in FIG. 2, shown with the balloon positioned to release and implant a stent in the side branch of a bifurcation.

### DESCRIPTION OF THE PREFERRED EMBODIMENTS

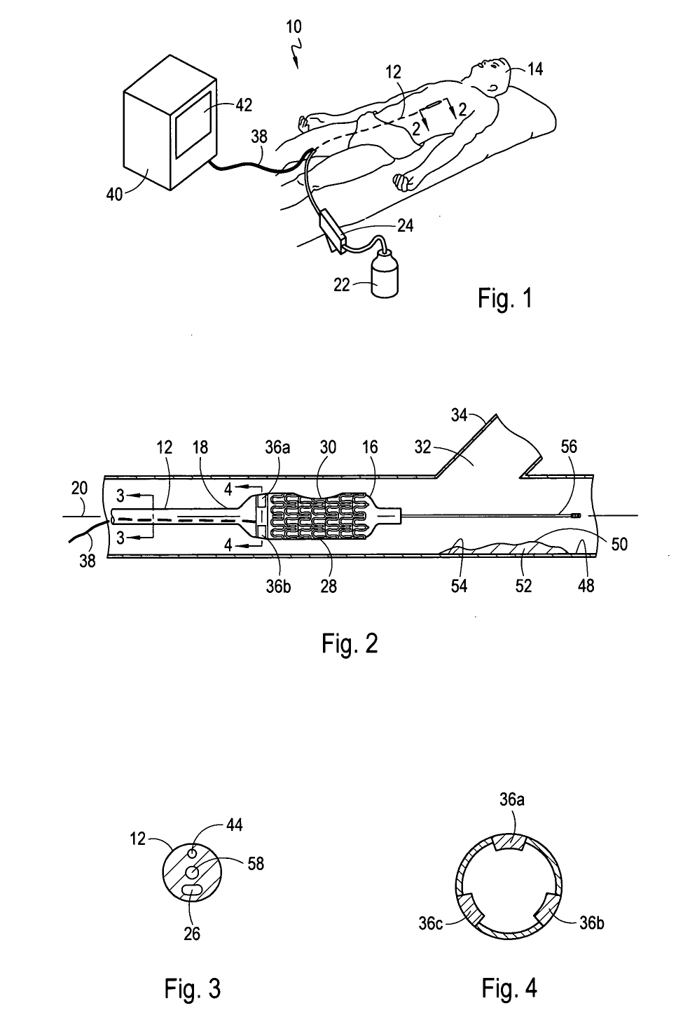

Referring initially to FIG. 1, a system for performing a percutaneous intervention is shown and generally designated 10. As shown, the system 10 includes a catheter 12, a distal portion of which has been inserted in the vasculature of a patient 14 using the femoral artery for access and advanced into the upper body of the patient 14. As detailed further below, the system 10 can be used to revascularize damaged or collapsed arteries and is specifically applicable for the treatment of lesions at or near a bifurcation, such as the aorta-ostium bifurcation. Although the system 10 is capable of performing a medical procedure in an upper body vessel such as the aorta, those skilled in the pertinent art will quickly recognize that the use of the system 10 as herein described is not limited to use in a specific vessel, but, instead can be used in vascular conduits to include veins, arteries and the chambers of the heart, as well as in other ductal systems throughout the human body.

Referring now to FIG. 2, a distal portion of the system 10 is shown to include an inflatable balloon 16 that is attached to the distal end 18 of catheter 12. For the system 10, the catheter 12 is typically elongated, made of a flexible polymeric material, and defines a substantially linear longitudinal axis 20 when the catheter 12 is straight. FIG. 2 further shows that the balloon 16 is typically elongated and can include a cylindrical shaped working section. Typically, the inflatable balloon 16 is made of a polymeric material and can be of the compliant, semi-compliant or non-compliant type construction.

For the system 10, the balloon 16 shown in FIG. 2 is reconfigurable on the catheter 12 between a first, deflated configuration and second, radially expanded (i.e. inflated) configuration. This reconfiguration can be accomplished by infusing the balloon 16 with a medical grade fluid, which may or may not contain a contrast agent, to expand the inflatable balloon 16. More specifically, as shown in FIG. 1, an inflation device, which for the embodiment shown consists of a fluid reservoir 22 in fluid communication with a hand-operable pump 24, can be activated to pass a medical grade fluid through the inflation lumen 26 (see FIG. 3) of the catheter 12 and into the balloon 16 to expand the inflatable balloon 16. Alternatively, a calibrated syringe (not shown), or any other inflation device known in the pertinent art to infuse a fluid through the catheter 12 and into the balloon 16, may be used.

FIG. 2 further shows that an expandable stent 28 is engaged with the balloon 16 and catheter 12 for movement therewith within the vasculature. For the system 10, the stent 28 is typically made of a metallic material and includes a periodic arrangement (i.e. pattern) of struts, as shown. When the stent 28 is radially expanded by an inflation of the balloon 16, the struts permanently deform causing the stent 28 to assume a new configuration having a larger internal radius. It is to be appreciated that the strut pattern shown in FIG. 2 is merely exemplary and those skilled in the pertinent art will recognize that stents having a wide variety of differing strut patterns are available and can be implanted using the system 10. The stent 28 shown in FIG. 2 also is formed with a lateral opening 30 that can be positioned at, and oriented toward, the ostium 32 of the side branch 34 of a bifurcation.

With cross-reference now to FIGS. 2 and 4, the system 10 includes provisions to precisely position the stent 28 for implantation at the correct (i.e. prescribed) location within a bifurcation. More specifically, the positioning system 10 includes a plurality of acoustic transceivers 36a-c, which, as shown, are uniformly distributed around the circumference of the distal end 18 of the catheter 12 and attached thereto. It can be further seen that each acoustic transceivers 36 is located at the proximal end of the stent 28. Moreover, the transceivers 36a-c are arranged as an annulus that is aligned with a plane that is substantially perpendicular to the catheter axis 20. Cross-referencing FIG. 2 with FIG. 1, it can be seen that the transceivers 36 are electrically connected over a multi-conductor wire 38 to an extracorporeally located controller 40, which can include a display 42. A wire lumen 44 is provided in the catheter 12 as shown in FIG. 3. For the system 10, the controller 40 can be used to selectively activate each transceiver 36 individually and, if desired, all of the transceivers 36 can be activated simultaneously. Although three transceivers 36 are shown for the system 10, it is to be appreciated that this number is merely exemplary and that more than three and as few as one transceiver 36 may be used.

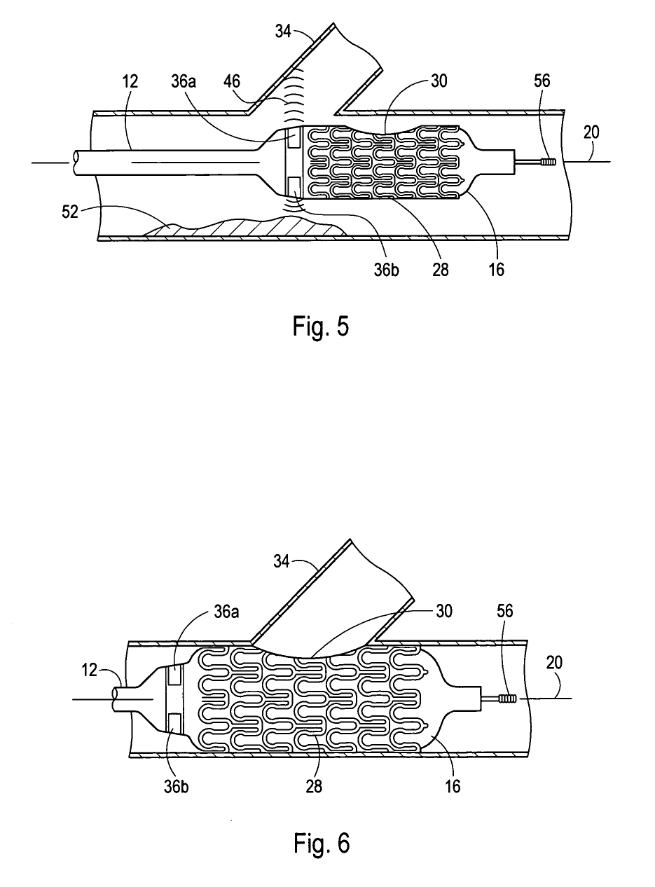

For the system 10, each transceiver 36 typically includes an acoustic transducer crystal which vibrates in response to an applied time-varying voltage. The vibration, in turn, produces an acoustic signal (which can be a pulse or a continuous wave) that is radiated from the respective transceiver 36. For the arrangement and positioning of transceivers 36 shown in FIGS. 2 and 4, each transceiver 36 radiates an acoustic signal (such as signal 46 shown in FIG. 5) that is directed substantially radially from the catheter axis 20. The frequency of the radiated acoustic signal 46 is dependent on the type of crystal used and, to a lesser extent, the applied excitation voltage. In one implementation of the present invention, transducer crystals are selected to radiate acoustic signals having ultrasonic frequencies. For the system 10, the acoustic signals 46 can be pulsed or continuous signals.

Once transmitted, the acoustic signals 46 are reflected with primary reflections occurring at interfaces which separate two dissimilar substances. Thus, primary reflections are expected at the vessel wall 48 and surfaces 50 of lesions 52, including the interface 54 between the lesion 52 and the vessel wall 48 (see FIG. 2). Each reflection generates an acoustic return signal that is received by the transmitting transceiver 36 and converted to an electrical return signal. The electrical return signals from each transducer crystal are sent over the wire 38 to the controller 40 where they are used to create an image for presentation on the display 42. The image can then be used to determine a spatial relationship between the proximal end of the stent 28 and the bifurcation. Specifically, the image can be used to evaluate the nature of the vessel wall, the location of the bifurcation side branch 34, and the presence/absence and size of any lesions, such as lesion 52.

### Operation

The operation of the system 10 can perhaps best be appreciated with initial cross-reference to FIGS. 1 and 2. As shown there, in a typical procedure, the stent 28 is first engaged with the balloon 16 and then inserted into and advanced through the vasculature of the patient 14 until the balloon 16 and stent 28 are located proximal the target bifurcation as shown in FIG. 2. In most cases, a guidewire 56 is used for this purpose. For the embodiment shown, the catheter 12 is an over-the-wire type catheter having a guidewire lumen 58 which typically extends the length of the catheter 12. Alternatively, the system 10 could be configured as a so-called monorail type catheter (not shown) having a guidewire lumen that is limited to a distal portion of the catheter 12 to allow the system 10 to be used as a so-called rapid exchange catheter.

With the stent 28 proximal the target bifurcation as shown in FIG. 2, one or more of the transceivers 36 are activated to produce an image on the display 42. Next, the catheter 12 is advanced through a small axial distance, and again, the transceiver(s) 36 are activated and the resultant image analyzed. This process can then be repeated. In some implementations of the system 10, the transceiver(s) 36 continue to radiate acoustic signals (pulsed or continuous) after initial activation and during movement of the catheter 12. FIG. 5 shows the transceivers 36 positioned at the side branch 34 of the bifurcation to produce a mapping of the bifurcation relative to the proximal end of the stent 28.

The images (i.e. mapping) can be evaluated to determine the geometry of the bifurcation and size and location of any lesions 52. This mapping can then be used in surgical planning, including the determination of a suitable stent 28 placement location, and, for example, to prescribe a subsequent side branch intervention. In addition, the mapping can be used to place the stent 28 at the prescribed location for implantation and orient the opening 30 of the stent 28 with the side branch 34. Once the operator is satisfied that the stent 28 is located at the correct position and oriented properly, the balloon 16 is inflated (see FIG. 6) to implant and release the stent 28. The balloon 16 is then deflated and removed from the patient 14 to complete the procedure.

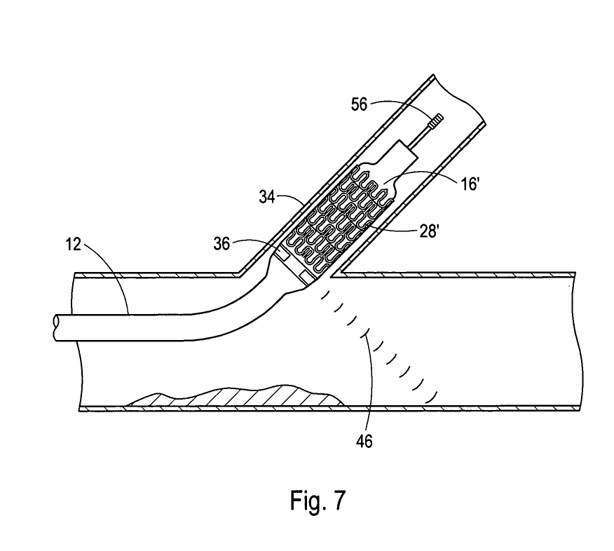

Although the above discussion has, for convenience, described the implantation of a stent 28 in a main branch of a bifurcation, it is to be appreciated that the system 10 can be used to implant stents in other places, for example, a side branch 34. This is shown in FIG. 7. Specifically, FIG. 7 shows the catheter 12 positioned to release and implant a stent 28′ in the side branch 34 of a bifurcation. Typically, as shown, a guidewire 56 is first routed into the side branch 34, past the sight where the stent 28′ is to be implanted. Next, the balloon 16′ and stent 28′ are advanced along the guidewire 56. During this advancement, one or more of the transceivers 36 are activated to generate acoustic signals 46 and produce an image on the display 42 (see FIG. 1). This image is then used to locate the stent 28′ in the side branch 34.

While the particular System for Stent Placement in a Vascular Bifurcation and corresponding methods of use as herein shown and disclosed in detail are fully capable of obtaining the objects and providing the advantages herein before stated, it is to be understood that they are merely illustrative of the presently preferred embodiments of the invention and that no limitations are intended to the details of construction or design herein shown other than as described in the appended claims.

1. A system for positioning a stent at a bifurcation in the vasculature of a patient, which comprises:
a catheter, said catheter being steerable for advancing said catheter through the vasculature;
an expandable means mounted on said catheter and engageable with the stent for selectively holding the stent on said catheter and releasing the stent therefrom;
a transceiver mounted on said catheter at a predetermined location relative to said engageable means;
a means for activating said transceiver to radiate a signal therefrom toward a vessel wall in the vasculature, and to receive said signal as a return signal after reflection thereof from the vessel wall; and
an extracorporeal means for evaluating said return signal to determine a spatial relationship between the stent and the bifurcation for positioning the stent at the bifurcation.

2. A system as recited in claim 1 further comprising a guidewire, said guidewire being pre-positioned in the vasculature across the bifurcation and said catheter being engageable with said catheter for steering said catheter through the vasculature.

3. A system as recited in claim 1 wherein said transceiver is an ultra-sonic transducer.

4. A system as recited in claim 3 wherein said ultrasonic transducer comprises a plurality of crystals for radiating a respective plurality of said signals and for receiving a respective plurality of said return signals, and further wherein said catheter defines an axis and said plurality of crystals are arranged as an annulus in a plane substantially perpendicular to the axis of said catheter.

5. A system as recited in claim 1 wherein the bifurcation is an aortic-ostium.

6. A system as recited in claim 1 wherein the expandable means is a balloon.

7. A system as recited in claim 6 wherein said balloon is moveable between a deflated configuration and an inflated configuration, and wherein the stent is released from said balloon when said balloon is moved from its inflated configuration to its deflated configuration.

8. A system for positioning a stent at a bifurcation in the vasculature of a patient, the stent having a distal end and a proximal end, said system comprising:
a catheter, said catheter being steerable for advancing said catheter through the vasculature;
an inflatable balloon mounted on said catheter and reconfigurable between a deflated configuration for engaging the stent for an advancement through the vasculature and an inflated configuration for releasing the stent from the balloon; and
a plurality of individually activatable, acoustic transducer crystals, each said crystal mounted on said catheter at an end of the stent for radiating an acoustic signal toward a vessel wall in the vasculature, receiving a reflected acoustic return signal therefrom, and converting said acoustic return signal to an electrical signal indicative of a spatial relationship between the stent and the bifurcation to allow the positioning of the stent at the bifurcation.

9. A system as recited in claim 8 further comprising a guidewire, said guidewire being pre-positioned in the vasculature across the bifurcation and said catheter being engageable with said catheter for steering said catheter through the vasculature.

10. A system as recited in claim 8 wherein said catheter defines an axis and said plurality of crystals are arranged as an annulus in a plane substantially perpendicular to the axis of said catheter.

11. A system as recited in claim 8 wherein the bifurcation is an aortic-ostium.

12. A method for positioning a stent at a bifurcation in the vasculature of a patient, which comprises the steps of:
providing a catheter having an expandable means mounted thereon, and a transceiver mounted thereon at a predetermined location relative to the expandable means;
engaging the stent with the expandable means to selectively hold the stent on the catheter;
steering the catheter through the vasculature with the stent thereon;
activating the transceiver to radiate a signal therefrom toward a vessel wall in the vasculature, and to receive the signal as a return signal after reflection thereof from the vessel wall; and
evaluating the return signal to determine a spatial relationship between the stent and the bifurcation for positioning the stent at the bifurcation.

13. A method as recited in claim 12 wherein said activating step is accomplished periodically.

14. A method as recited in claim 12 wherein said steering step further comprises the steps of:
pre-positioning a guidewire in the vasculature across the bifurcation; and
engaging the catheter with the guidewire to steer the catheter through the vasculature.

15. A method as recited in claim 12 wherein the transceiver is an ultra-sonic transducer.

16. A method as recited in claim 12 wherein the bifurcation is an aortic-ostium.

17. A method as recited in claim 12 wherein the expandable means is a balloon.

18. A method as recited in claim 17 wherein the balloon is moveable between a deflated configuration and an inflated configuration, and wherein said engaging step is accomplished with the balloon in its deflated configuration.

19. A method as recited in claim 18 further comprising the step of releasing the stent from the balloon when the stent is properly positioned at the bifurcation.

20. A method as recited in claim 19 wherein said releasing step is accomplished by moving the balloon from its inflated configuration to its deflated configuration.
